(19) United States
(12) Patent Application Publication
(10) Pub. No.: US 2014/0316097 A1
(43) Pub. Date: Oct. 23, 2014

(54) METHOD AND APPARATUS FOR RECOVERING LACTIDE FROM POLYLACTIDE OR GLYCOLIDE FROM POLYGLYCOLIDE

(71) Applicant: Rainer Hagen, Berlin (DE)

(72) Inventor: Rainer Hagen, Berlin (DE)

(73) Assignee: UHDE INVENTA-FISCHER GMBH, Berlin (DE)

(21) Appl. No.: 14/238,800

(22) PCT Filed: Aug. 17, 2012
(86) PCT No.: PCT/EP2012/066065

§ 371 Date: Jun. 10, 2014

(30) Foreign Application Priority Data
Aug. 19, 2011 (EP) 11006825.1

(51) Int. Cl. C07D 319/12; C08G 63/08

(52) U.S. Cl. CPC C07D 319/12; C08G 63/08
USPC 528/272; 549/274; 422/162; 422/134

(57) ABSTRACT

The present invention relates to a process for recovering lactide from polylactide (PLA) or glycolide from polyglycolide (PGA), in which, in a first step, PLA or PGA is contacted with a hydrolysing medium and hydrolytically degraded to oligomers. In a further step, a cyclising depolymerisation of the oligomers obtained in the first step is effected to give lactide or glycolide. In addition, the present invention relates to an apparatus based on the combination of a hydrolysis apparatus with a depolymerisation reactor, with which the above-described process can be performed. The core of the process according to the invention is a partial hydrolysis of the polymeric materials originally used in combination with a cyclising depolymerisation.

The present invention relates to a method for recovering lactide from polylactide (PLA) or for recovering glycolide from polyglycolide (PGA), wherein in a first step PLA or PGA is brought into contact with a hydrolyzing medium and is hydrolytically degraded to oligomers. A cycling depolymerization of the oligomers obtained in the first step to form lactide or glycolide takes place in a further step. In addition, the present invention relates to an apparatus which is based on the combination of a hydrolysis apparatus with a depolymerization reactor and with which the previously described method can be carried out. The core of the method in accordance with the invention is a partial hydrolysis of the originally used polymer materials in combination with a cyclizing depolymerization.

It is known to split PLA by a complete hydrolysis with water or with lactic acid containing water into lactic acid which can again be converted into PLA after a corresponding purification in known ways.

These methods have the disadvantage that partial racemization occurs during the complete hydrolysis. In this process, lactic acid is produced from PLA, which is composed, for example, of 98% L-lactic acid units and 2% D-units, said lactic acid having a D-portion which is considerably larger than the 2% to be expected.

M. Faisal et al. (Asian Journal of Chemistry Vol. 19, No. 3 (2007), p. 1714-1722) investigated the hydrolysis of PLA in the temperature range between 160 and 350° C. for 3 h. High yields of lactic acid are obtained at high temperatures or with long dwell times; conditions at which the L-lactic acid also partially racemizes to D-lactic acid.

M. Yagihashi and T. Funazukuri (Ind. Eng. Chem. Res. 2010, 49, p. 1247-1251) used diluted aqueous soda lye for the hydrolysis of PLA in the temperature range between 70 and 180° C. and compared with pure water. No racemization was observed at reaction times in the range from 20-60 min. On implementation in a technical process, the sodium content of the produced lactic acid has to be removed with a high effort.

A further disadvantage of all processes which work with complete hydrolysis of the PLA to lactic acid with respect to the method in accordance with the invention is that, in a process for manufacturing PLA from lactic acid comprising dehydration, polycondensation to the oligomer, depolymerization to the lactide, lactide purification, ring opening polymerization of the lactide to PLA, which is used almost exclusively on an industrial scale, the purified lactic acid recycled in this manner has to run through all the named process steps. The dehydration and polycondensation consume energy for the separation of water, increase the D-portion in the polycondensate of the L-lactic acid by partial racemization and require apparatus capacity for the carrying out.

A method is known from DE 196 37 404 B4 which converts PLA into dilactide without a preceding hydrolysis by depolymerization. The very high catalyst amount at 3-7% and a very high temperature at up to 300° C. are disadvantages. The depolymerization of high-molecular PLA to lactide is very slow in comparison with PLA-oligomer due to the low end-group concentration so that the reaction speed has to be increased by a high catalyst concentration and a high temperature.

A method is described in WO 2010/118954 which dissolves polymer blends containing PLA in a solvent, which removes the non-dissolved polymer portions, which depolymerizes PLA hydrolytically to lactic acid or to a derivative thereof and which purifies the product. The solvent which has to be removed from the product and recovered is disadvantageous with this method.

WO 2011/029648 claims a method for recycling a mixture of PLA of different contents of optical isomers which is linked with a separation of these isomers from the stage of the lactide. The PLA is dissolved in a solvent and subsequently depolymerized by transesterification in this method. The transesterification product is separated from the solvent and is then converted to raw lactide by cyclizing depolymerization. The purification of the raw lactide includes a separation of the meso-lactide. The solvent which has to be separated and recovered is again disadvantageous in this method. The dwell time in the depolymerization is very long.

The Japanese patent application JP 2009-249508 describes a recycling method which combines partial hydrolysis with depolymerization of the produced oligomer to the lactide. It has the advantage of reducing the racemization during the hydrolysis. It is disadvantageous that the hydrolysis occurs in the discontinuous process with water vapor in the solid PLA. A large-scale technical process can be realized economically with difficulty in this manner.

There is therefore a need for a continuous recycling method for PLA which reduces the effort for energy and apparatus and simultaneously decreases the partial racemization.

It is thus the object of the present invention to provide a recovery method for lactide or glycolide from their respective polymers which is improved in comparison with the previously described methods and to provide a corresponding apparatus for carrying out this method.

This object is achieved by the features of claim 1 with respect to the method and by the features of claim 15 with respect to the apparatus. The respective dependent claims in this respect represent advantageous further developments.

In accordance with the invention, a method for recovering lactide from polylactide (PLA) or for recovering glycolide from polyglycolide (PGA) is provided, wherein
a) in a first stage or in a first step, PLA or PGA is brought into contact with a hydrolyzing medium in the melt and is hydrolytically degraded to PLA oligomers having a number average molar mass Mₙ between 162 and 10,000 g/mol (measured by acid-base titration of the carboxyl groups) or to PGA oligomers having a number average molar mass Mₙ between 134 and 10,000 g/mol (measured by acid-base titration of the carboxyl groups); and
b) in a further stage, the PLA oligomers or PGA oligomers are subsequently subjected to a cyclizing depolymerization to lactide or glycolide.

If the process in accordance with the invention is compared with known recycling processes of PLA which work with complete hydrolysis of the waste to lactic acid, the following advantages can be named:
The partial hydrolyzate in accordance with the method in accordance with the invention no longer has to be degraded by polycondensation to the oligomer like lactic acid from a complete hydrolysis reaction. This saves energy which is required for the vacuum evaporation of the water chemically formed during the polycondensation.
At the same time, the partial hydrolyzate does not undergo the racemization which is not only observed on the building up of the oligomer from lactic acid, but rather also in the complete hydrolysis of the PLA to lactic acid.

Since the PLA quality is reduced by racemization, this is a quality advantage or—with existing separation possibilities between L-lactic acid and D-lactic acid or between L-lactide, D-lactide and meso-lactide—a saving of apparatus effort and energy for separating these components.
The method in accordance with the invention in particular provides the possibility at a plurality of points of separating foreign substances which can be present as solids in the molten PLA and can be retained by a melt filter before the hydrolysis:
   After the hydrolysis to the oligomer, a highly fluid melt is present from which the solid foreign substances which are taken along and their solid decomposition products can be separated in a less complex manner by filtration. A disposable filter having a wire mesh or a nonwoven fabric is suitable for this purpose in which the loaded filter sheets can be replaced by fresh ones and can be removed from the filter apparatus without interrupting the melt flow. Filter apparatus are also suitable in which the filter medium can be regenerated by backflushing. Filtration after the hydrolysis is an option. It can be made use of or omitted depending on the kind of contaminants present in the waste products.
   The second possibility for the separation of foreign substances is the cyclizing depolymerization of the hydrolyzate. >95% of the hydrolyzate are there converted into lactide which is evaporated in vacuum and exits the reactor. What remains is—as also in the manufacturing process of the fresh PLA—an oligomer residue which also contains the foreign substances of low volatility or solid foreign substances or their decomposition products in addition to the products of the thermal oligomer degradation. This residue leaves the process and serves as a natural outlet for contaminants.
   All the contaminants which are more volatile than the lactide, e.g. produced by thermal decomposition of foreign substances, remain in the following lactide condensation in the vapor phase and are sucked up by the vacuum pumps together with the secondary products typical for PLA such as water and lactic acid and so exit the process. Residues which possibly remain in the raw lactide are removed in the subsequent lactide purification. Foreign substances or their decomposition products which react with the raw lactide have to be removed before the cyclizing depolymerization, either in the prepurification of the waste products or by filtration after the hydrolysis.

The following definitions are understood under the respective terms in the claims and in the following description:

Lactide: Cyclic ester of 2 lactic acid molecules which can occur in the form of the pure L-lactide ((S,S)-lactide), D-lactide ((R,R)-lactide) or meso-lactide ((S,R)-lactide) or (in most cases) in the form of a mixture of at least two of these components. The cyclizing depolymerization produces raw lactide which can contain linear oligomers, higher cyclic oligomers and residues of lactic acid and water in addition to the named isomers of the lactide. L-lactide is to be understood as the cyclic ester of two L-lactic acid units; D-lactide as the cyclic ester of two D-lactic acid units; meso-lactide the cyclic ester of a D-lactic acid unit and of an L-lactic acid unit. Racemic lactide (rac-lactide) is a 1:1 mixture of L-lactide and D-lactide.

Glycolide: cyclic ester of two glycolic acid molecules;

Polylactide (PLA): polymer of lactic acid units, e.g. produced by ring-opening polymerization of the L-lactide, D-lactide or meso-lactide or a mixture of two or three of these lactides. It can also be a mixture of polymers from the named pure or mixed lactides. (Pure) PLA is here also understood as PLA which comprises stereocomplex crystallites or consists entirely thereof. PLA as a rule has a number average molecular weight >10,000 g/mol.

Polyglycolide (PGA): Polymer of glycolic acid units, e.g. produced by ring-opening polymerization of the glycolide. As a rule, PGA has a number average molecular weight >10,000 g/mol.

PLA waste products or PGA waste products: The invention is in particular suitable for the recycling of PLA waste products or PGA waste products which arise from the processing of PLA or PGA, e.g. cut-off marginal strips of PLA films or PGA films or sprues of injection molded parts. These waste products are normally of single origin, i.e. they do not contain any other plastics. The invention is, however, also suitable for used packaging, textiles, components of technical plastics of single-origin PLA or PGA which is contaminated with foreign substances from use which cannot be completely separated by preceding mechanical separation methods. Examples for such foreign substances are sugar from sugary drinks or edible oils which have penetrated into the bottle wall by migration and which cannot be completely removed by washing the used bottle.

Finally, the invention is suitable for PLA waste products or for PGA waste products which were acquired by preceding mechanical separation methods such as sorting, washing, floating, comminution, screening from a mixture or a compound with other substances, e.g. paper, metal, glass or other plastics and still contain residues of these foreign substances. Examples include textiles from mixed fibers or from multicomponent threads, multilayer films having one or more layers of PLA, PGA or composites containing PLA or PGA. A further example is the foreign polymer fraction which is produced from comminution and sorting of used plastic bottles. A complete mechanical separation of foreign substances is here normally not possible for economic reasons.

Residues of foreign substances do not represent any obstacle for the method in accordance with the invention under certain circumstances. Foreign substances originate from the processing, the use or the waste disposal of the packaging, textiles or engineering plastics. They include in this respect e.g. food residues or beverage residues, coloring agents, labels, adhesives, residues of paper, metal, glass or plastics. A foreign substance portion in the waste products below around 5% is preferred, particularly preferably below 1%. Foreign substances do not represent a problem which are not volatile under the conditions of cyclizing depolymerization (vacuum, temperature) or which form non-volatile intermediate products or volatile decomposition products with a boiling point of <150° C. at atmospheric pressure. Non-volatile foreign substances or decomposition products collect in the residue of the cyclizing depolymerization. Volatile decomposition products below the stated boiling point cannot contaminate the lactide which has formed if they do not condense in a similar temperature range to lactide. Foreign substances which themselves react with lactide or which form decomposition products in the depolymerization which react with lactide, should preferably be removed before they are subjected to the method in accordance with the invention.

Methods of lactide purification or glycolide purification: The method in accordance with the invention utilizes known processes for the purification of raw lactide or raw glycolide from cyclizing depolymerization. Such processes are e.g. rectification (EP 0 893 462 B1), crystallization from the melt (U.S. Pat. No. 3,621,664 or WO 2007/114) or from a solvent. The disclosure content of these documents with respect to the mentioned cleaning methods is also made the subject of this patent application by reference.

In addition to the separation of residues of water, lactic acid, linear and higher cyclic oligomers, the separation or setting of the content of meso-lactide, L-lactide and D-lactide in the pure lactide is a necessary element of the lactide purification.

The quality of the PLA which is prepared from lactide and which is produced using the method in accordance with the invention depends on its content of D-lactide units and meso-lactide units. As the content of D-lactic acid units in the PLA predominantly made up of L-lactide units increases, its melting point, thermal shape stability, crystallization speed and degree of crystallization fall. If this content of D-units in the PLA exceeds 6-8%, the polymer is amorphous; the melting point coincides with the glass transition temperature of e.g. 55° C. The same applies to meso-lactide and L-lactide in a PLA predominantly made up of D units.

The L-lactic acid unit content and D-lactic acid unit content of the PLA waste products cannot be predicted in a PLA recycling process. To make the quality of the end product independent of the composition of the waste products, a separation or a concentration adjustment of the optical lactide isomers is preferred. In this way, the lactide composition in the raw substance of the ring-opening polymerization and thus in the PLA can be set as desired.

Meso-lactide can be removed from the melt or its content set in L-lactide or D-lactide both by distillation methods and by crystallization. D-lactide and L-lactide can be separated by crystallization from the melt or from a solution.

PLA melt: PLA has a melting temperature between 40° C. and 170° C. depending on the isomeric purity of the lactide of which it is made up. PLA containing stereocomplex crystallites has a melting temperature of up to 230° C. The method in accordance with the invention uses partial hydrolysis and cyclizing depolymerization in the molten liquid phase to make the method continuous in a comfortable and economic manner. The basic requirement for the method is therefore a flowable melt. It must be anticipated with PLA waste products due to the heterogeneous composition that they contain PLA with very different melting temperatures.

Hydrolyzing medium: In accordance with the invention, this includes water, lactic acid, glycolic acid or a mixture of water and lactic acid and mixtures of water and glycolic acid. Pure lactic acid admittedly does not effect hydrolysis with PLA, but rather transesterification. The effect—the degradation to PLA oligomers—is, however, the same as with hydrolysis. A distinction is therefore superfluous for the purposes of this invention. However, lactic acid in industrially available quantities always contains water and catalytically promotes hydrolysis. The term “lactic acid” used in accordance with the invention thus includes small portions of water. Other acids containing water also promote hydrolysis, but subsequently have to be removed from the product since they, unlike lactic acid, disturb the depolymerization to the lactide or its polymerization to PLA.

Partial hydrolysis: Like all polyesters, PLA or PGA is also accessible to hydrolytical splitting of the polymer chains. What has to be avoided in the polymerization and processing, is used in the method in accordance with the invention to directly degrade the polymer down to a desired molar mass. Although the hydrolysis of the PLA already runs at temperatures below the melting point, the dwell times required for it or the required water concentration in the polymer are very high. A technical process is advantageous when it reaches the goal in short dwell times, which results in small construction sizes of the apparatus. The process in accordance with the invention furthermore utilizes the lowest possible amount of water for the hydrolysis with the goal of reaching only exactly the desired molar mass. Excess water is thus avoided which has to be removed again after the end of the hydrolysis reaction with an effort of energy and apparatus. Hydrolysis with a considerably larger water amount furthermore results in greater partial racemization (JP 2009-249508; M. Faisal et al, Asian Journal of Chemistry Vol. 19, No. 3 (2007), p. 1714).

The desired mean molar mass (number average) after the hydrolysis amounts to between 162 and 10,000 g/mol with lactide oligomers or to between 134 and 10,000 g/mol with glycolide oligomers, preferably between 400 and 2000 g/mol in each case. This molar mass range is particularly suitable for the subsequent depolymerization to the lactide or to the glycolide as is known from instructions on the technical design of lactide production or glycolide production.

Cyclizing depolymerization: This reaction is the reverse reaction of the ring-opening polymerization which is used in almost all industrial processes for the production of the PLA from lactide or of the PGA from glycolide. It is unwanted in the finished PLA or PGA because it results in a molar mass reduction, and thus in a deterioration of the product properties, during processing. It is used in the production of lactide to produce a raw lactide from the oligomer obtained by polycondensation of the lactic acid which forms the starting material for the ring opening polymerization after purification.

The partial hydrolytic degradation to the respective oligomers starting from the polymer materials PLA or PGA to the stated mean molecular weight, which is carried out in the first stage, in this respect takes place by a corresponding choice of the respective parameters such as the amount of the medium to be hydrolyzed with respect to the polymer materials as well as the temperature and the pressure. The corresponding parameters can be set and determined using simple experiments.

A preferred embodiment provides that the hydrolyzing medium is selected from the group comprising water, lactic acid, glycolic acid, mixtures of water and lactic acid and mixtures of water and glycolic acid. If pure lactic acid or pure glycolic acid is used, it is understood in accordance with the invention that these pure substances contain at least traces of water.

In a further advantageous embodiment, the number average molecular weight of the PLA oligomers or PGA oligomers obtained in step a) amounts to between 400 and 2,000 g/mol. The number average molecular weight is in this respect determined by acid-base titration of the carboxyl groups of the respective oligomers.

It is further advantageous if 50 mmol to 10 mol, preferably 100 mmol to 5 mol, in particular 0.5 mol to 3 mol, of hydrolyzing medium is added per 1 kg of the mass of PLA or PGA.

Preferred parameters which are set during the hydrolytic degradation in step a) are in this respect:
α) a temperature of 130 to 300° C., preferably of 150 to 250° C., particularly preferably of 190 to 230° C.;
β) a pressure of 5 to 500 bar, preferably from 10 to 300 bar, particularly preferably from 20 to 200 bar; and/or
γ) a dwell time of 0.1 to 50 min, preferably of 1 to 15 min, particularly preferably of 1 to 5 min.

It is further advantageous if a catalyst is added to the oligomers before the cyclizing depolymerization in step b), in particular in a concentration of 0.01 to 50 mmol/kg of oligomers, further preferably of 0.1 to 10 mmol/kg of oligomers.

Preferred polymer starting materials in this respect originate in part or in full from waste material which arises, for example, from reject material in the polymerization of lactide or glycolide. In this respect, before the charging in step a), the PLA waste products or PGA waste products are
α) purified of foreign substances by sorting, washing and other separation methods so that the portion of foreign substances and/or contaminants preferably amounts to less than 5% by weight, further preferably less than 1% by weight, with respect to the mass of PLA or PGA; and/or
β) comminuted so that the maximum dimension of the obtained communited waste products amounts to 15 mm.

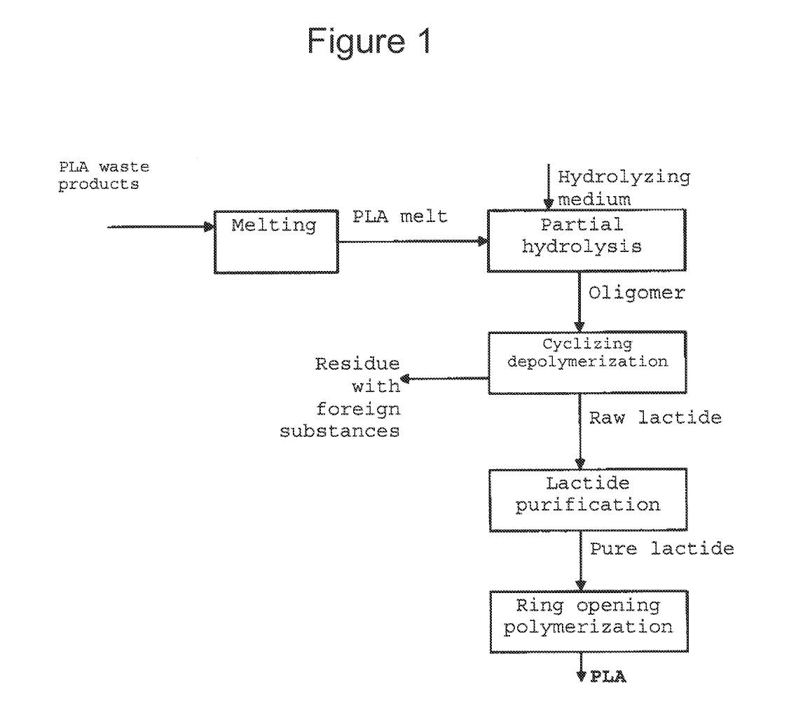

It is further advantageous if the oligomers obtained in step a)
α) are added to lactic acid or to glycolic acid charged in a polycondensation stage, with the respective oligomers being produced from lactic acid or glycolic acid;
β) are added to a polycondensation stage of lactic acid or glycolic acid, with the respective oligomers being produced from lactic acid or glycolic acid; and/or
γ) are added to the oligomers obtained from a polycondensation stage of lactic acid or glycolic acid
   and the oligomer mixture thus obtained is supplied to the cyclizing depolymerization (step b)). This embodiment is in particular shown in FIG. 1.

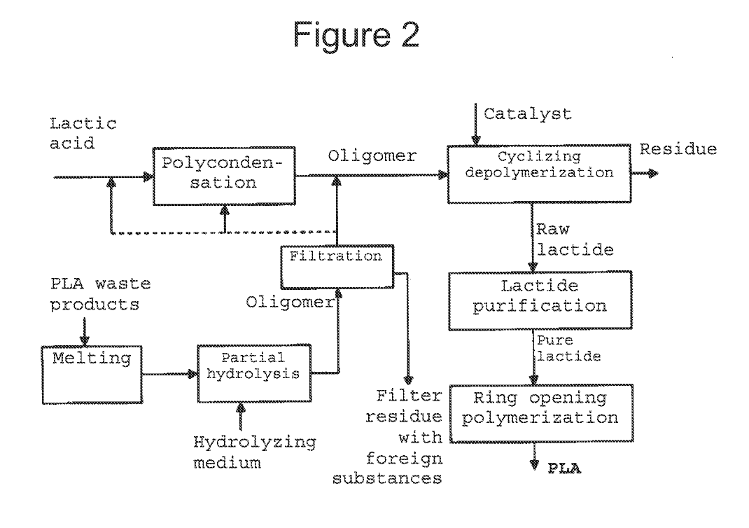

The previously described preferred embodiment thus provides that the oligomers first produced by hydrolytic degradation are used for further process steps in the manufacture of oligomers of lactic acid or glycolic acid. This can take place, for example, by addition of the oligomers obtained in step a) (see in this respect, for example, FIG. 2 and associated statements) to lactic acid or glycolic acid, i.e. before oligomerization of these monomers or also directly in such a condensation stage to the respective oligomers. Equally, the oligomers obtained from step a) can also be combined with oligomers of lactic acid or glycolic acid originating from further process lines.

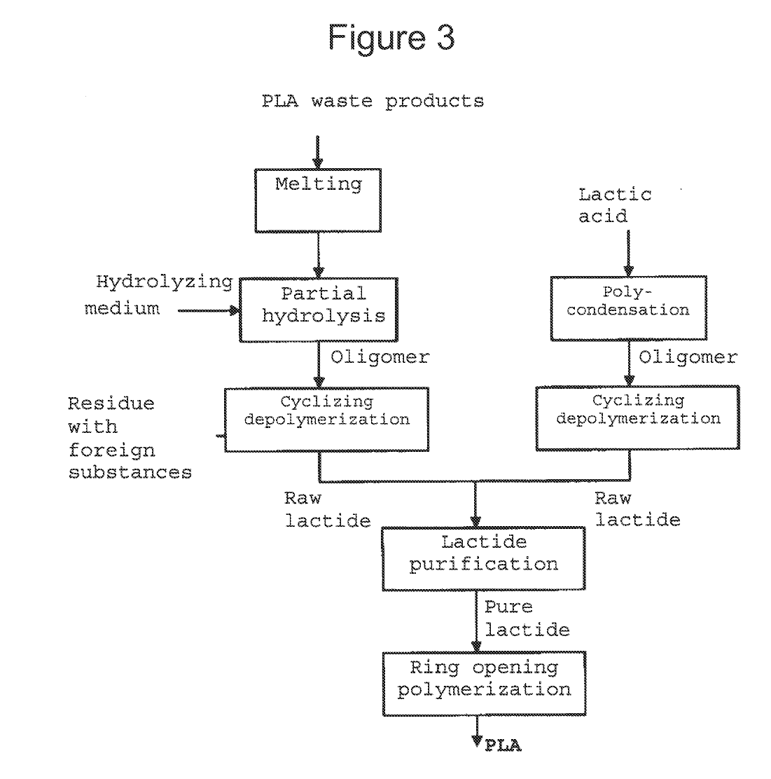

Provision can equally be made that the lactide or glycolide obtained by the cyclizing depolymerization (step b)) is mixed with lactide or glycolide which was obtained by polycondensation of lactic acid or glycolic acid to oligomers and cyclizing depolymerization of these oligomers (see in this respect, for example, FIG. 3 and associated statements).

It can moreover be advantageous to break down the lactide obtained in step b) into different fractions. The lactide fractions obtained in this process can in this respect be enriched with or depleted of meso-lactide and/or enriched with or depleted of L-lactide, D-lactide and/or meso-lactide. The content of the respective lactide fraction is in this respect compared with the lactide content of the lactide fraction obtained directly from stage b). The separation of the respective fractions can in this respect take place by fractioning distillation of by the initially described separation and purification processes.

The lactide obtained after stage b) is in this respect in particular—in comparison with the lactide directly obtained from stage b)—
α) separated into a fraction depleted by meso-lactide and a fraction enriched with meso-lactide; or
β) is separated into a fraction enriched with L-lactide, a fraction enriched with D-lactide and/or a fraction enriched with meso-lactide, with the concentration of these components in the fraction preferably amounting to ≧50% by weight, preferably ≧90% by weight, particularly preferably ≧98% by weight.

It is further preferred if
α) before step b), the oligomers and/or
β) after step b), the obtained lactide or glycolide is/are subjected to a purification, in particular a distillation purification and/or a recrystallization.

In accordance with an additionally preferred embodiment, the PLA or PGA is charged into the stage a) in the molten state or is melted during stage a). The contacting of the PLA or PGA with the hydrolyzing medium in stage a) can thus therefore already take place in the molten state; it is equally also possible that the contacting takes place before the melting of the PGA or PLA. The partial hydrolysis is in this respect preferably carried out in the molten state.

The method is in particular suitable for continuous process management.

In accordance with the invention, a method is equally provided for producing PLA or PGA, wherein the method starts from lactide or glycolide and these monomers are converted into PLA or PGA in a ring-opening polymerization. In this respect, it is characteristic and in accordance with the invention that at least some, or the totality, of the lactide or glycolide used was produced in accordance with a previously described recovery process.

The invention furthermore relates to an apparatus for the continuous recovery of lactide from PLA or of glycolide from PGA which comprises
a) an apparatus for melting PLA or PGA and/or an apparatus for supplying a PLA melt or PGA melt, as well as
b) a hydrolysis apparatus arranged downstream of the apparatus for melting and/or of the apparatus for supplying a PLA melt or PGA melt; and
c) a depolymerization reactor arranged downstream of the hydrolysis apparatus.

A hydrolysis apparatus in which the partial hydrolysis of the PLA or PGA is carried out is thus a central element of the apparatus in accordance with the invention.

In this respect an apparatus for melting PLA or PGA or an apparatus for supplying a PLA melt or PGA melt into the hydrolysis solution is disposed upstream of the hydrolysis apparatus.

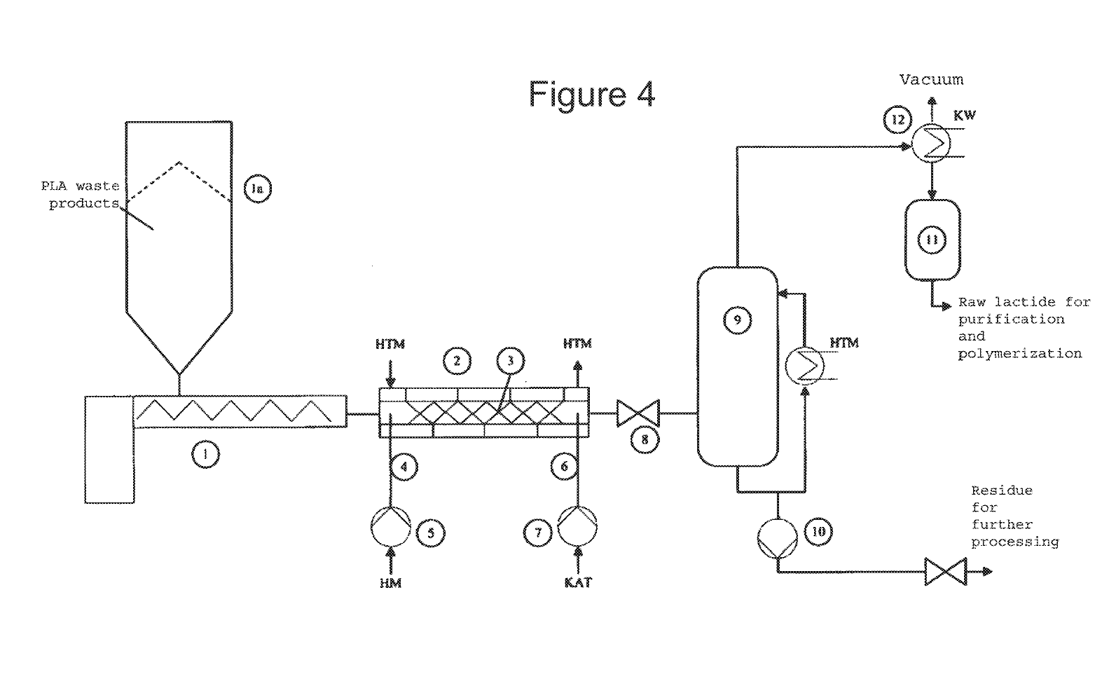

Downstream of the apparatus for melting and/or of the apparatus for supplying a PLA melt or PGA melt, the apparatus in accordance with the invention thus comprises a hydrolysis apparatus for carrying out a partial hydrolysis of the PLA melt or PGA melt to PLA oligomers or PGA oligomers which is in communication with the apparatus for melting and/or the apparatus for supplying a PLA melt or PGA melt via a melt line for the melt, wherein the hydrolysis apparatus is disposed upstream of the depolymerization reactor (see FIG. 4 in this respect).

In accordance with the invention, PLA or PGA is thus first melted in a melting apparatus and this melt is supplied to the hydrolysis apparatus or a melt is already supplied to the hydrolysis apparatus, wherein the partial hydrolysis of the PLA or PGA takes place in the hydrolysis apparatus. The PLA oligomers or PGA oligomers arising in this process are then supplied to the depolymerization reactor in which a cyclizing depolymerization to lactide or glycolide is carried out.

In this respect, it is preferred if
a) the apparatus for melting is an extruder or a melting grating;
b) the apparatus for supplying a PLA melt or a PGA melt is a melt line and/or a melt pump;
c) the hydrolysis apparatus has a heatable pipe stretch which preferably has heatable or non-heatable static mixing elements; and/or
c) the depolymerization reactor is designed as a circulation evaporator, a falling film evaporator, a thin film evaporator or as a combination of two or three of these constructions.

The hydrolysis apparatus further advantageously has
a) an input possibility for a hydrolyzing medium which preferably opens into the melt supply of the hydrolysis apparatus; and/or
b) an input possibility for a depolymerization catalyst which preferably opens into the melt outflow of the hydrolysis apparatus.

It is equally preferred if the depolymerization reactor has an extractor apparatus via which non-converted melt can be removed.

The depolymerization reactor can have an extractor for lactide vapors or glycolide vapors which is arranged at the head side and which opens into a condensation apparatus for lactide vapors or glycolide vapors.

A further advantageous embodiment provides that the condensation apparatus is disposed downstream of a supply apparatus for the produced lactide or glycolide and/or a purification apparatus for lactide or glycolide.

A polycondensation reactor for producing PLA oligomers or PGA oligomers by polycondensation of lactic acid or glycolic acid can be arranged between the hydrolysis apparatus and the depolymerization reactor, with the outlet of the hydrolysis apparatus opening in front of, in or after the polycondensation reactor.

The outlet of the depolymerization reactor can run together with the outlet of a reactor which produces PLA oligomers or PGA oligomers by means of polycondensation of lactic acid or glycolic acid in a first stage and which produces lactide or glycolide from these PLA oligomers or PGA oligomers by means of cyclizing depolymerization in a second stage.

The present invention further comprises an apparatus for producing PLA or PGA by ring-opening polymerization of lactide or glycolide which contains a previously described apparatus for the continuous recovery of lactide from PLA or glycolide from PGA, an apparatus for purifying the lactide and a downstream ring-opening polymerization apparatus.

The present invention will be described in more detail with reference to the following embodiments and to the enclosed Figures without restricting the invention to the specific embodiments shown there.

There is shown:

FIG. 1 the general routine of a method in accordance with the present invention starting from PLA waste products up to the production of fresh PLA by ring-opening polymerization;

FIG. 2 a method of producing PLA with the infeed of partially hydrolyzed PLA waste products;

FIG. 3 a method of producing PLA with the infeed of raw lactide produced from PLA waste products; and

FIG. 4 a schematic representation of an apparatus in accordance with the invention for carrying out the method.

FIG. 1 shows the basic scheme of the routine of a method in accordance with the invention using PLA waste products as an exemplary starting material. A melting of the PLA waste products takes place in a first step; the PLA melt thus produced is admixed with a hydrolyzing medium and is subsequently hydrolyzed, whereby a partial degradation of the PLA polymer chain takes place and PLA oligomers are thus produced. A subsequent cyclizing depolymerization takes place after this partial hydrolysis; a raw lactide is obtained in this respect. Under certain circumstances, a residue with foreign substances is produced as a secondary product. The raw lactide thus produced can be subjected to an optional lactide purification, for example a distillation purification or a purification by recrystallization. In the example shown in FIG. 1, the pure lactide thus produced is directly subsequently subjected to a ring-opening polymerization, whereby new polylactide can be produced.

The method in accordance with the invention can be carried out in a recycling plant (FIG. 1) which comprises apparatus for melting, hydrolysis, cyclizing depolymerization, lactide purification and ring-opening polymerization. Overall, a complete PLA plant therefore has to be provided for this method which has the same extent, measured by the number of process steps, as a PLA plant which starts from lactic acid.

The pure lactide from such a recycling plant can also be transported as a raw material for ring-opening polymerization to a production plant for virgin PLA and can be used for the PLA production there. The apparatus for the ring-opening polymerization can be dispensed with in this manner.

FIG. 2 describes a method of producing PLA with the infeed of partially hydrolyzed PLA waste products which were produced in accordance with the method of the present invention. The step of preparing PLA waste products shown in the lower left hand branch in FIG. 2 substantially corresponds to the process management already explained in FIG. 1. In this respect, PLA waste products are melted in a first stage and are supplied to a partial hydrolysis while adding a hydrolyzing medium. The oligomers thus produced can be liberated from foreign substances by filtration, for example. The purified oligomers are combined with the oligomers which originate from the polycondensation of lactic acid. Alternatively and/or additionally to this, the oligomers from the partial hydrolysis of the PLA waste products cans also already be admixed to the lactic acid used as the reactant and can be charged in the polycondensation stage or can be charged directly into the polycondensation stage of the lactic acid. These possibilities are indicated by the dashed arrows in FIG. 2. The oligomers thus obtained are supplied to the cyclizing depolymerization, wherein a raw lactide is produced. The cyclizing depolymerization is preferably carried out while adding a catalyst; remaining residues are removed from this stage. After an optional purification, for example by distillation or crystallization, the pure lactide can again be supplied to a ring-opening polymerization and can be converted to PLA. A further purification of the pure lactide by separation of optical isomers is, however, also equally conceivable.

It is equally advantageous to operate the method in accordance with the invention as part of an individual PLA method and a plant in accordance with the invention as a part of a PLA production plant. In this case, the PLA production plant only has to be expanded by a side line having apparatus for melting and for the partial hydrolysis of the PLA waste products (FIG. 2). After the infeed of the partial hydrolyzate before, in or after the polycondensation, it replaces some of the raw material lactic acid. No further process stages up the PLA are therefore required for the recycled material, but the existing ones are also used. It is obvious that this possibility provides economic advantages.

FIG. 3 shows a further variant of the process management for producing PLA which makes use of the method in accordance with the invention for the recovery of lactide from PLA waste products. In two parallel branches of the process management, raw lactide is produced, on the one hand, by partial hydrolysis and cyclizing depolymerization of PLA waste products (see FIG. 3, left hand branch); this process management runs identically with the first three steps of the process management shown in FIG. 1. In a second branch (see FIG. 3, right hand branch), the production of raw lactide equally takes place; however, from lactic acid, with a polycondensation with subsequent cyclizing depolymerization taking place. Both raw lactide flows can be subjected in combined form to a purification, wherein pure lactide is obtained. This pure lactide can be directly subsequently supplied to a polymerization to PLA.

This embodiment of the invention is e.g. of advantage when larger quantities of foreign substances remain in the non-converted residue during the cyclizing depolymerization or merge into the lactide vapors which would disturb the PLA process in the main line. In this variant, the raw lactide from the PLA waste products is added to the main line before the lactide purification so that the apparatus of the lactide purification and the following ring-opening polymerization of the main line are also used for the recycled material. Substantial apparatus savings also result in this variant. It must be mentioned for reasons of completeness that the raw lactide from the PLA waste products can also be added before, in or after the polycondensation step of the main line.

FIG. 4 shows an apparatus for carrying out the method in accordance with the invention with reference to an exemplary embodiment. The abbreviations here stand for:

HM: hydrolyzing medium (water, lactic acid, mixture of water and lactic acid, etc.)

KAT: catalyst

HTM: heat carrier medium

The apparatus in accordance with FIG. 4 comprises a single-screw extruder 1 for melting the comminuted, presorted and prepurified waste products which are taken from a storage container. A pressure-tight pipe stretch 2 which is heatable from the outside and which is inwardly equipped with static mixing elements 3 is connected to the extruder outlet. At the inlet of the pipe stretch, a capillary 4 of stainless steel serves e.g. the supply of water for the partial hydrolysis. The capillary is provided with a metering pump 5 which meters demineralized water or diluted lactic acid into the PLA melt against the melt pressure. A second capillary 6 with a metering pump 7 is arranged at the end of the pipe stretch. It serves the metering of the liquid or dissolved catalyst for the depolymerization.

A pressurizing valve 8 is arranged at the end of the pipe stretch and the melt is expanded through it into the following depolymerization reactor which is at vacuum. This reactor is designed as a circulating evaporator 9 in this example. This stage is equipped with an extractor apparatus 10 through which the non-converted melt is discharged.

The lactide vapors arising by the depolymerization reaction are extracted from the reaction stage and are liquefied in a condensation apparatus 12. The liquid raw lactide enters into a collector tank 11 and from there into the lactide purification. It is converted to polymerizable pure lactide there.

The apparatus described here is an embodiment for illustrating the method. The description is therefore in no way to be understood as a restriction of the method. The underlying method of the invention can also be carried out in apparatus modified in a variety of ways, as mentioned in the method description.

Preferred variants of the process management in accordance with the invention will be illustrated with reference to the following embodiments.

PLA waste products are melted after sorting and after a suitable mechanical prepurification. An exactly metered quantity of water, lactic acid or a mixture of the two is added to the melt and effects a hydrolysis to an oligomer. The oligomer is subjected to a cyclizing depolymerization which produces a raw lactide as the product. After purification of this lactide using known processes, it can be polymerized to PLA without losses of properties. The method can be particularly advantageously used in combination with an apparatus which is associated with a plant for producing new PLA product.

The technical design of the method in accordance with the invention takes place e.g. and preferably such that the waste products are purified mechanically according to known processes by sorting, washing and separating foreign substances so that the portion of foreign substances amounts to less than 5% by mass, preferably less than 1% by mass. After comminution to a maximum dimension of 15 mm, which can take place before or after the separation of foreign substances depending on the type and properties of the waste products, the waste products are continuously supplied to an extruder and melted.

The extruder can be designed as a single-shaft machine, a twin-shaft machine or a multishaft machine. A single-shaft extruder is preferred which is completely filled with melt and can build up a melt pressure. A degassing for the separation of moisture is not necessary during the melting since the subsequent hydrolysis anyway requires water. A machine with degassing provides advantages when gases or other volatile substances than water arise in the melting which may disturb the following process. Instead of an extruder, a melting grating is also suitable which likewise provides the possibility of degassing. The melting grating is an inexpensive alternative which can, however, only be considered with very small foreign substance portions when there is no risk of separation of contaminants from the melt which could clog the grating.

The extruder is adjoined by a hydrolysis apparatus which substantially comprises a heated pipe section which provides the melt with the dwell time required for the hydrolysis reaction. In order further to mix and homogenize the melt during the hydrolysis, the pipe stretch can be equipped with a static mixer. Mixing elements of the type SMX from the Sulzer corporation are e.g. suitable for this purpose. The diameter of the pipe stretch increases with larger throughputs. From a diameter of around 15 mm onward, the pipe stretch contains pipes for leading through a liquid heat carrier in addition to the static mixing elements for a better heat supply. These pipes can also be coiled or folded in the form of static mixing elements (e.g. Sulzer SMR) and then do not require any additional static mixing elements.

A melting pump, e.g. a gear pump, can be arranged between the extruder and the melt line. This is necessary when the extruder cannot apply the required roughing pressure for the hydrolysis apparatus. A melting pump for a pressure increase is obligatory in the event a melting grating is used.

Water, lactic acid or mixes of the two are suitable for the hydrolysis. While water requires the design of the hydrolysis apparatus for a pressure of at least 30 bar to remain liquid at the reaction temperature and to avoid evaporation, corrosion-resistant materials are required at the infeed point and at the melt-contacted parts disposed downstream on the use of lactic acid. It is advantageous to preheat water, lactic acid or their mixtures prior to the infeed into the PLA melt to the melt temperature to prevent freezing of the melt at the infeed point.

The amount of water or lactic acid required for the hydrolysis is selectively metered at pressure into the extruder, between the extruder and the hydrolysis apparatus or between the melt pump and the hydrolysis apparatus. The mean molar mass after the end of the partial hydrolysis is preferably set via the quantity of the added hydrolyzing medium. In this case, the melt is in reaction equilibrium after the hydrolysis reaction, with the mean molar mass also being in chemical equilibrium. An “overshoot” of the reaction with the consequence of a mean molar mass lower than the desired one is precluded in this way. With water as the hydrolysis medium, the added quantity is at up to 100 g H₂O/kg PLA, preferably at around 20 g H₂O/kg PLA. The pressure amounts to >30 bar, upwardly limited only by the pressure strength of the components, as a rule around 200 bar, and thus by the costs of the apparatus.

It is also possible to set the desired molar mass by limiting the dwell time and the melt temperature during the hydrolysis reaction. In this case, however, larger fluctuations of the molar mass in the hydrolysis product can be expected.

In a variant of the invention, the hydrolyzing medium is fed into the melting extruder. A melting temperature in the upper range of the given span is preferably selected in this variant to keep the dwell time required for the hydrolysis low and already to lead the hydrolysis reaction to the desired molar mass in the extruder. In this case, no separate hydrolysis apparatus is required and the melt can be directly expanded and supplied to the depolymerization reactor.

After the hydrolysis reaction, a filtration of the melt can be of advantage which withholds contaminants and foreign substances which are present as particles having dimensions above around 50 micrometers (e.g. carbonized particles). This is inter alia of use when foreign substances which can react with lactide or their decomposition products can be removed there.

The melting temperature at the end of the melting process is selected so that all PLA particles are melted and no further temperature increase is required for the hydrolysis so that it is completely over within a dwell time between 1 and 30 minutes, preferably between 1 and 5 minutes. The melting temperature during the hydrolysis reaction is between 150° and 250° C., preferably between 190° C. and 230° C.

After the hydrolysis, an oligomer of the lactic acid is present which is highly fluid at the melt temperature. It does not differ in molar mass from the oligomer which arises by polycondensation of the lactic acid in the first step of the lactide production in ring-opening polymerization. Differences include the content of polymerization catalyst and stabilizer as well as optionally of foreign substances and their decomposition products from the PLA waste products. This lactic acid oligomer is converted into lactide in the second step of the method in accordance with the invention. This reaction is the reverse reaction of the ring-opening polymerization which is used in almost all industrial processes for the production of the PLA from lactide. The same catalysts are therefore also active—both in the cyclizing depolymerization and in the ring-opening polymerization. As a rule, PLA and thus PLA waste products contain tin compounds as the polymerization catalyst. Depending on the origin and quality of the PLA waste products, catalyst for the cycling depolymerization is therefore added between 0 and 20 mmol/kg; in most cases, however, 0 to 10 mmol/kg is sufficient. Organic tin compounds, e.g. tin(II)-octoate, are preferably suitable as the catalyst. However, all other catalysts known for the ring-opening polymerization of lactides are also suitable.

The method in accordance with the invention will be explained in more detail with reference to the following examples. The following analytical methods were used for this purpose:

Carboxyl end groups of the PLA hydrolyzate, molar mass of the PLA oligomers:

The PLA oligomer is dissolved in acetone. After the addition of methanol, the solution is titrated with 0.1 N benzyl alcohol KOH solution. The end point is detected potentiometrically. The number average of the molar mass can be calculated from the carboxyl end group concentration (“COOH”), measured in mmol/kg, using the equation Mn=10⁶/COOH.

Carboxyl groups in the lactide:

The lactide sample is dissolved in methanol and is subsequently titrated in the same manner as in the carboxyl end group determination in the PLA oligomer.

Determining the intrinsic solution viscosity:

The weighed polymer quantity is dissolved in a defined volume of chloroform. The throughput time of the solution and of the pure solvent is measured in an Ubbelohde capillary viscometer which is in a thermostatized water bath set to 30° C.+/−01° C. The quotient of both is the relative solution viscosity. It is converted into the intrinsic viscosity (I.V.) using the single-point method of J. Dorgan et al., J. Polym. Sci.: Part B: Polym. Physics, Vol. 43, p. 3100-3111, (2005). The I.V. is in relation with the weight average M_{w} of the molar mass of the polymer which is described by the so-called Mark-Houwink equation. For the substance pair PLA/chloroform, the equation reads (J. Dorgan, loc.cit.):

I.V.=K*Mw*a, where K=1.53*10⁻⁴ dl/g,

a=0.759

Optical isomers of the lactide:

The lactide sample is dissolved in a mixture of 90/10 ml/ml n-hexane/ethanol. The dissolved components are separated by HPLC on a chiral column and are analyzed using a UV detector at 223 nm.

D-portion in the lactic acid and PLA:

A sample of PLA or of a PLA oligomer is hydrolyzed in a boiling process with 1-N soda lye at the backflow and is neutralized after cooling. The neutralized sample is mixed with 3 millimolar copper sulfate solution in a ratio 1/9 ml/ml and is separated with HPLC on a stereospecific column into the components which are subsequently analyzed by a UV detector at a wavelength of 238 nm.

A lactic acid sample is directly dissolved in the 3 millimolar copper sulfate solution and is analyzed with HPLC as described.

Residual monomer content in PLA

A balanced PLA sample is dissolved in a defined volume of chloroform. 100 μm of this solution is injected into a GPC system which is equipped with a cross-linked polystyrene acid of different pore size and with a refractive index detector. Chloroform serves as the eluent.

The dilactide peak is identified by its retention time. The substance quantity is determined from its surface with the aid of a previously recorded calibration curve and is converted into a dilactide concentration using the weighed-in PLA.

### EXAMPLE 1

This example shows the basic suitability of the combination of hydrolysis and cyclizing depolymerization for the conversion of PLA into raw lactide.

30 g PLA with an I.V. of 1.80 dl/g, corresponding to a weight-average molar mass of 230,000, is admixed with a water quantity of 0.70 g in a laboratory pressure container of stainless steel which is lined with PTFE and is closed. The container is introduced into a circulation air drying cabinet which is maintained at a temperature of 190° C. After 6 hours, the autoclave is removed from the drying cabinet and is cooled by being left to stand at room temperature. The container is opened and the produced viscous mass is removed and analyzed.

A carboxyl end group concentration of 1250 is found by titration which corresponds mathematically to a molar mass of 800 Da.

20 g of the viscous mass is admixed in a three-neck glass flask with stirrer with 0.184 mg Sn-octoate, dissolved in toluene. After application of a vacuum of 5 mbar, the glass flask is brought by an oil bath from the outside to 220° C. The temperature in the oil bath is measured. The development of vapors starts while stirring; they are led off out of the flask, condensed in a vertical coil cooler at vacuum and are collected as yellowish crystals in a cooled glass flask.

The depolymerization is stopped after 4.0 hours by removing the three-neck glass flask from the heated oil bath. The vapor development comes to a stop at this time. A blackish brown residue, of 1.8 g, remains in the flask and is a glassy solid at room temperature. After the vacuum is broken by N₂, the collector flask is removed from the condenser and the content is weighed and analyzed. There is 17.8 g of a raw lactide with a carboxyl group number of 350 mmol/kg. The sample contains 95.2% L-lactide and 4.8% meso-lactide according to the HPLC analysis.

### EXAMPLE 2

The example shows the effect of the method in accordance with the invention. The extruder 1 is set in an apparatus in accordance with FIG. 4 such that it takes in and melts 1.5 kg/h comminuted PLA waste products. The waste products show a mean I.V. of 1.64 dl/g (chloroform, 30° C.).

The pressurization valve 8 at the end of the pipe stretch 2 is set to 30 bar. The temperature of the melt at the outlet of the extruder is 210° C. 26.9 g/h demineralized water is urged through the capillary 4 into the melt at the start of the pipe stretch with the aid of the metering pump 5. A sample of the melt after the pressurization valve 8 shows a carboxyl end group concentration of 960 mmol/kg.

The metering pump 7 conveys 300 ppm Sn as Sn-octoate via the capillary 6 into the hydrolyzed melt at the end of the pipe stretch 2. A melt temperature of 220° C. is set in the depolymerization reactor 9. 70 g/h liquid residue is removed from the reactor and 1,410 kg/h raw lactide from the condensation apparatus 13. A concentration of 5.3% meso-lactide is measured at the raw lactide by means of HPLC.

A sample of the raw lactide of 2000 g results in pure lactide having a carboxyl group concentration of 5 mmol/kg and a portion of meso-lactide of 0.4% after recrystallization from toluene as a solvent and after filtration and drying of the crystallizate.

The pure lactide quantity is melted in a laboratory autoclave with agitator under nitrogen as a protective gas, is mixed with 85 ppm Sn as Sn-octoate and is polymerized for 3 hours after increasing the melt temperature to 180° C. The PLA melt is then removed from the stirring container as a strand, is quenched and solidified by drawing through a water bath and is pelletized. 950 g granulate is collected and is homogenized by mixing. The residual monomer content, determined by gel permeation chromatography, amounts to 5.2%. A sample of the granulate is liberated from the residual monomer by dissolving with chloroform and precipitating with isopropanol and is analyzed after drying. The I.V. amounts to 1.82; the D-portion to 0.5%. The properties of the recycled PLA are thus at a level which corresponds to a new product.

1-24. (canceled)

25. A method of recovering lactide from polylactide (PLA) or glycolide from polyglycolide (PGA), wherein:
a) PLA or PGA is brought into contact with a hydrolyzing medium in the melt and is hydrolytically degraded to PLA oligomers having a number average molar mass Mₙ between 162 and 10,000 g/mol (measured by acid-base titration of the carboxyl groups) or to PGA oligomers having a number average molar mass Mₙ between 134 and 10,000 g/mol (measured by acid-base titration of the carboxyl groups); and
b) the PLA oligomers or PGA oligomers are subsequently subjected to a cyclizing depolymerization to lactide or glycolide.

26. The method of claim 25, wherein the hydrolyzing medium is selected from water, lactic acid, glycolic acid, mixtures of water and lactic acid, and mixtures of water and glycolic acid.

27. The method of claim 25, wherein, in step a) PLA or PGA is hydrolytically degraded to PLA oligomers or PGA oligomers with a number average molar mass Mₙ between 400 and 2,000 g/mol (measured by acid-base titration of the carboxyl groups).

28. The method of claim 25, wherein 50 mmol to 10 mol of the hydrolyzing medium is added per kg of the mass of PLA or PGA.

29. The method of claim 25, wherein, during the hydrolytic degradation in step a):
α) a temperature of the melt of 130 to 300° C. is set;
β) a pressure of 5 to 500 bar is set; and/or
γ) a dwell time of the melt of 0.1 to 50 min is observed.

30. The method of claim 25, wherein, before the cyclizing depolymerization in step b), a catalyst is added to the oligomers.

31. The method of claim 25, wherein the PLA or PGA originates at least in part or in full from waste material, and wherein, before charging in step a), the PLA waste products or PGA waste products
α) are purified of foreign substances by a mechanical separation method so that the portion of foreign substances and/or contaminants amounts to less than 5% by weight with respect to the mass of PLA or PGA; and/or
β) are comminuted so that the maximum dimension of the obtained comminuted waste products amounts to 15 mm.

32. The method of claim 25, wherein the oligomers obtained in stage a)
α) are added to lactic acid or glycolic acid which are charged into a polycondensation stage, wherein the respective oligomers are produced from the lactic acid or glycolic acid;
β) are added to a polycondensation stage of lactic acid or glycolic acid, with the respective oligomers being produced from lactic acid or glycolic acid; and/or
γ) are added to the oligomers obtained from a polycondensation stage of lactic acid or glycolic acid
and the oligomer mixture thus obtained is supplied to the cyclizing depolymerization (stage b)).

33. The method of claim 25, wherein the lactide or glycolide obtained by the cyclizing depolymerization (stage b)) is mixed with lactide or glycolide which was obtained by polycondensation of lactic acid or glycolic acid to oligomers and cyclizing depolymerization of these oligomers.

34. The method of claim 25, wherein the lactide obtained after stage b) is—in comparison with the lactide directly obtained from stage b)—
α) separated into a fraction depleted by meso-lactide and a fraction enriched with meso-lactide; or
β) is separated into a fraction enriched with L-lactide, a fraction enriched with D-lactide and/or a fraction enriched with meso-lactide, with the concentration of these components in the fraction amounting to ≧50% by weight.

35. The method of claim 25, wherein:
α) before step b), the oligomers and/or
β) after step b), the obtained lactide or glycolide
is/are subjected to a purification and/or a recrystallization.

36. The method of claim 25, wherein the PLA or PGA is charged into stage a) in a molten state or is melted during stage a).

37. The method of claim 25, wherein the method is carried out continuously.

38. A method of manufacturing PLA or PGA, wherein lactide or glycolide is converted to PLA or PGA in a ring-opening polymerization, which includes a recovery method of claim 25, is first carried out and at least some or the totality of the lactide or glycolide utilized for the ring-opening polymerization was produced by the recovery method.

39. An apparatus for the continuous recovery of lactide from PLA or glycolide from PGA, comprising
a) an apparatus for melting PLA or PGA
and/or an apparatus for supplying a PLA melt or PGA melt;
b a hydrolysis apparatus for carrying out a partial hydrolysis of the PLA melt or PGA melt to PLA oligomers or PGA oligomers which is arranged downstream of the apparatus for melting and/or of the apparatus for supplying a PLA melt or a PGA melt and which has an input possibility for a hydrolyzing medium and/or an input possibility for a depolymerization catalyst; and
c) a depolymerization reactor arranged downstream of the hydrolysis apparatus.

40. The apparatus of claim 39, wherein the hydrolysis apparatus is in communication with the apparatus for melting and/or with the apparatus for supplying a PLA melt or a PGA melt via a melt line for the melt.

41. The apparatus of claim 39, wherein
α) the apparatus for melting is an extruder or a melting grating;
β) the apparatus for supplying a PLA melt or a PGA melt is a melt line and/or a melt pump;
γ) the hydrolysis apparatus has a heatable pipe stretch; and/or
δ) the depolymerization reactor is designed as a circulation evaporator, a falling film evaporator, a thin-film evaporator or as a combination of two or three of these constructions.

42. The apparatus of claim 41, wherein:
α) the input possibility for a hydrolyzing medium opens into the melt supply of the hydrolysis apparatus; and/or
β) the input possibility for a depolymerization catalyst opens into the melt outflow of the hydrolysis apparatus.

43. The apparatus of claim 41, wherein the depolymerization reactor has an extraction apparatus via which the non-converted melt can be discharged.

44. The apparatus of claim 39, wherein the depolymerization reactor has an extractor for lactide vapors or glycolide vapors which is arranged at the head side and which opens into a condensation apparatus for lactide vapors or glycolide vapors.

45. The apparatus of claim 44, wherein a storage apparatus for lactide or glycolide and/or a purification apparatus for lactide or glycolide is/are arranged downstream of the condensation apparatus.

46. The apparatus of claim 40, wherein a polycondensation reactor for producing PLA oligomers or PGA oligomers by polycondensation of lactic acid or glycolic acid is arranged between the hydrolysis apparatus and the depolymerization reactor, wherein the outlet of the hydrolysis apparatus opens before, in, or after the polycondensation reactor.

47. The apparatus of claim 39, wherein the outlet of the depolymerization reactor runs together with the outlet of a reactor which produces PLA oligomers or PGA oligomers by means of polycondensation of lactic acid or glycolic acid in a first stage and lactide or glycolide from these PLA oligomers or PGA oligomers by means of cyclizing depolymerization in a second stage.

48. An apparatus for producing PLA or PGA by ring-opening polymerization of lactide or glycolide, comprising an apparatus for the continuous recovery of lactide from PLA waste products or glycolide from PGA waste products from the apparatus of claim 39 as well as a ring-opening polymerization apparatus disposed downstream.
